(19) United States
(12) Patent Application Publication
(10) Pub. No.: US 2005/0287163 A1
(43) Pub. Date: Dec. 29, 2005

(54) Mycoplasma polypeptides

(75) Inventors: Walter H. Hsu, Ames, IA (US); Theresa F. Young, Carlsbad, CA (US); Richard F. Ross, Ames, IA (US); En-Min Zhou, Ames, IA (US)

Correspondence Address: FISH & RICHARDSON P.C., PO BOX 1022, MINNEAPOLIS, MN, 55440-1022, US

(73) Assignee: Iowa State University Research Foundation, Inc., Ames, IA (US)

(21) Appl. No.: 10/509,926

(22) PCT Filed: Apr. 4, 2003
(86) PCT No.: PCT/US03/10305

§ 371 Date: Jul. 29, 2005

Related U.S. Application Data
(60) Provisional application No. 60/370,344, filed on Apr. 5, 2002.

(51) Int. Cl. A61K 39/02; G01N 33/554; G01N 33/569; C07H 21/04; C07K 14/35

(52) U.S. Cl. 424/190.1; 435/7.32; 435/69.3; 435/252.3; 435/471; 530/350; 530/388.4; 536/23.7

(57) ABSTRACT

The invention provides methods and materials related to mycoplasma. For example, the invention provides mycoplasma polypeptides having the ability to increase calcium release from cells (e.g., porcine ciliated tracheal cells) as well as antibodies that bind to such mycoplasma polypeptides. In addition, the invention provides methods for identifying inhibitors of mycoplasma-induced calcium release from porcine ciliated tracheal cells.

### STATEMENT AS TO U.S. FEDERALLY SPONSORED RESEARCH

Funding for the work described herein was provided by the U.S. federal government, which may have certain rights in the invention.

### BACKGROUND

1. Technical Field

The invention relates to mycoplasma polypeptide preparations as well as antibody preparations having antibodies against mycoplasma polypeptides.

2. Background Information

Mycoplasmas are a large group of diverse prokaryotic species comprising the class Mollicutes. Mycoplasmas lack a cell wall, have a remarkably small genome, are phylogenically related to gram-positive eubacteria, and are the smallest known self-replicating organisms (Razin, Microbiol. Rev., 49:419-455 (1985); Razin, FEMS Microbiol. Lett., 79:423-432 (1992); and Razin and Jacobs, J. Gen. Microbiol., 138:407-422 (1992)). The surface of the mycoplasmas is clearly critical for the interaction of these organisms with their host cells (Freundt and Edward. 1979. Classification and taxonomy. p. 1-42. In M. F. Barile and S. Razin (eds.), The Mycoplasmas. Academic press, New York, N.Y.; Rogers et al., Proc. Natl. Acad. Sci. USA, 82:1160-1164 (1985); and Woese et al., J. Mol. Evol., 21:305-316 (1984-1985)).

Mycoplasma hyopneumoniae (Mhyo) is the etiological agent of mycoplasmal pneumonia of swine, which continues to cause significant economic losses to swine producers. This organism is an extracellular pathogen, and it colonizes in the respiratory epithelium of the pig. The role of M. hyopneumoniae infection in association with other swine respiratory pathogens has gained increased importance (Ross, R F, 1999. Mycoplasmal diseases, p. 495-509. In B. E. Straw, S. D'Allaire, W. L. Mengeling, and D. J. Taylor (eds), Diseases of Swine. Iowa State University Press, Ames, Iowa). For instance, M. hyopneumoniae potentiates porcine reproductive and respiratory syndrome virus-induced pneumonia (Thacker et al., J. Clin. Microbiol., 37:620-627 (1999)). M. hyopneumoniae induces pneumonia by first damaging the ciliated epithelial cells of the trachea, bronchi, and bronchioles (Debey et al., Am. J Vet. Res., 53:1705-1710 (1992); Mebus and Underdahl, Am. J Vet. Res. 38:1249-1254 (1977); and Tertyshnikova and Fein, Cell Calcium, 21:331-344 (1997)). However, the mechanisms underlying the M. hyopneumoniae-induced ciliary damages or loss of cilia are not well-understood. Recently, a tracheal epithelial cell model was developed, which enabled us to study the pathogenesis of M. hyopneumoniae 91-3 (Zhang et al., Infect. Immun., 62:4367-4373 (1994)).

The adherence of M. hyopneumoniae to ciliated epithelium is necessary to induce colonization of the organism, which results in the loss of cilia (Mebus and Underdahl, Am. J Vet. Res., 38:1249-1254 (1977); Zhang et al., Infect. Immun., 62:1616-1622 (1994); and Zhang et al., Infect. Immun., 63:1013-1019 (1995)). Thus, the adherence of mycoplasma to its host cells is an important initial step in the pathogenesis of mycoplasmal diseases. The adherence process is mainly mediated by receptor-ligand interactions (Zhang et al., Infect. Immun., 62:4367-4373 (1994); Zhang et al., Infect. Immun., 62:1616-1622 (1994); Zhang et al., Infect. Immun., 63:1013-1019 (1995); and Zielinski and Ross, Am. J. Vet. Res., 54:1262-1269 (1993)). Consistent with this concept are the observations that virulent strains of M. hyopneumoniae adhere to cilia of tracheal tissue in vitro, in contrast to avirulent strains of M. hyopneumoniae (Young et al., Vet. Microbiol., 71:269-279 (1999)).

### SUMMARY

The invention involves methods and materials related to mycoplasma polypeptide preparations having the ability to increase calcium release from porcine ciliated tracheal cells. Such polypeptide preparations can be used to generate polypeptide fragments having the ability to block mycoplasma-induced calcium release and can be used to generate antibodies having the ability to bind mycoplasma polypeptides. The invention also provides antibodies that bind to mycoplasma polypeptides. Such antibodies can be used to inhibit mycoplasma-induced calcium release and can be used to differentiate between pathogenic and non-pathogenic mycoplasma In addition, the invention provides methods for identifying inhibitors of mycoplasma-induced calcium release from porcine ciliated tracheal cells. Such inhibitors can be used to protect swine from developing mycoplasmal pneumonia and can be used to treat swine having mycoplasmal pneumonia

In general, one aspect of the invention features a substantially pure polypeptide, where the polypeptide increases calcium release from porcine ciliated tracheal cells, and where the molecular weight of the polypeptide is between about 30 kDa and about 150 kDa. The polypeptide can be a mycoplasma polypeptide. The polypeptide can be obtained from pathogenic Mycoplasma hyopneumoniae. The polypeptide can be about 80 percent pure or about 90 percent pure. The molecular weight of the polypeptide can be about 30, 60, 65, 90, or 120 kDa. The polypeptide can be a tryptic fragment. The molecular weight of the polypeptide following a tryptic digest can be about 35 kDa or 50 kDa.

In another aspect, the invention features a substantially pure antibody capable of binding a polypeptide, where the polypeptide increases calcium release from porcine ciliated tracheal cells, and where the molecular weight of the polypeptide is between about 30 kDa and about 150 kDa. The antibody can be a monoclonal antibody. The antibody can be a mouse antibody. The polypeptide can be a tryptic fragment. The polypeptide can be a mycoplasma polypeptide. The polypeptide can be obtained from pathogenic Mycoplasma hyopneumoniae. The antibody can be about 80 percent pure or about 90 percent pure.

Another aspect of the invention features a method for inducing an immune response in a mammal, where the immune response is against a mycoplasma polypeptide. The method includes administering a substantially pure mycoplasma polypeptide to the mammal under conditions wherein the mammal produces antibodies against the polypeptide, where the polypeptide increases calcium release from porcine ciliated tracheal cells, and wherein the molecular weight of the polypeptide is between about 30 kDa and about 150 kDa. The mammal can be a mouse, rabbit, or pig.

Another aspect of the invention features a method for binding an antibody to a polypeptide, where the polypeptide increases calcium release from porcine ciliated tracheal cells, and wherein the molecular weight of the polypeptide is between about 30 kDa and about 150 kDa. The method includes (a) obtaining an antibody capable of binding the polypeptide, and (b) contacting the antibody with the polypeptide under conditions wherein the antibody binds the polypeptide. The antibody can be a monoclonal antibody. The antibody can be a mouse antibody. The polypeptide can be a mycoplasma polypeptide.

Another aspect of the invention features a method for identifying an inhibitor of mycoplasma induced calcium release from porcine ciliated tracheal cells. The method includes (a) contacting cells (e.g., porcine ciliated tracheal cells) with a mycoplasma polypeptide and a test compound, where the polypeptide increases calcium release from porcine ciliated tracheal cells, and wherein the molecular weight of the polypeptide is between about 30 kDa and about 150 kDa, and (b) determining whether the test compound inhibits the cells from releasing calcium, where inhibition of calcium release from the cells by the test compound indicates that the test compound is the inhibitor. The test compound can be a protease or antibody.

In another embodiment, the invention features a method for identifying an inhibitor of calcium release from cells (e.g., porcine ciliated tracheal cells) induced by a mycoplasma polypeptide, where the polypeptide increases calcium release from porcine ciliated tracheal cells, and where the molecular weight of the polypeptide is between about 30 kDa and about 150 kDa. The method includes (a) contacting cells (e.g., porcine ciliated tracheal cells) with a mycoplasma polypeptide pretreated with a test compound, and (b) determining whether the test compound inhibits the cells from releasing calcium, where inhibition of calcium release from the cells by the test compound indicates that the test compound is the inhibitor. The test compound can be a protease or antibody.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 contains three graphs plotting [Ca²⁺]ᵢ response in ciliated porcine tracheal cells. Data are representative traces showing the effects of (a) pathogenic M. hyopneumoniae strain 91-3 (n=10, a total of 47 cells), (b) nonpathogenic M. hyopneumoniae (n=6, a total of 18 cells), and (c) M. flocculare (n=8, a total of 24 cells). The protein concentration for all three mycoplasma preparations was 300 μg/mL. The arrow indicates when the mycoplasma was administered.

FIG. 2 is a bar graph plotting the increase in [Ca²⁺]ᵢ over basal levels for the indicated treatments. PMH represents pathogenic M. hyopneumoniae strain 91-3; NPMH represents nonpathogenic M. hyopneumoniae; and MF represents M. flocculare. Data represent the mean ±SE. Intact M. hyopneumoniae 91-3 was administered at 30 (n=18 tracheal cells in 6 experiments), 100 (n=16 cells in 7 experiments), and 300 μg/mL (n−47 cells in 10 experiments). M. flocculare (n=24 cells in 8 experiments) and nonpathogenic M. hyopneumoniae (n=18 cells in 6 experiments) were administered with 300 μg/mL. Asterisks indicate significant differences from other treatments (P<0.05).

FIG. 3 contains four graphs plotting [Ca²⁺]ᵢ response in ciliated porcine tracheal cells inoculated with M. hyopneumoniae strain 91-3. Data are representative traces showing the effect of (a) Ca²⁺ free medium (n=5 cells), (b) pretreatment with thapsigargin (TG; 1 μM) for 30 minutes (n=5 cells), (c) U-73122 (2 μM; n=5 cells) for 100 seconds, and (d) U-73343 (2 μM; n=5 cells) on M. hyopneumoniae-induced increase in [Ca²⁺]ᵢ. The arrow indicates when the intact mycoplasma (300 μg/mL) was administered.

FIG. 4 contains four graphs plotting [Ca²⁺]ᵢ response in ciliated porcine tracheal cells inoculated with mastoparan 7 (Mas 7) or M. hyopneumoniae after pretreatment with pertussis toxin (PTX; 100 ng/mL) for 3 hours. Data are representative traces for (a) M. hyopneumoniae controls (n=9 cells), (b) M. hyopneumoniae treated with PTX (n=11 cells), (c) Mas 7 (10 μM) controls (n=9 cells), and (d) Mas 7 treated with PTX (n=9 cells).

FIG. 5 is a diagram of a proposed model of M. hyopneumoniae-ciliated tracheal cell interactions. Rc=receptor; ER=endoplasmic reticulum.

FIG. 6 contains four graphs plotting [Ca²⁺]ᵢ response in ciliated porcine tracheal cells inoculated with Mhyo membranes. Each trace indicates the [Ca²⁺]ᵢ changes in each tracheal cell. The arrows indicate when administration occurred. (A) The membrane preparation (100 μg/mL) increased [Ca²⁺]ᵢ. (B) Digestion with proteinase K blocked the membrane-induced increase in [Ca²⁺]ᵢ. The membrane (100 μg/0.1 mL PBS) was incubated with proteinase K (2 μg) at 37° C. for 8 hours before it was applied to tracheal cells in 0.9 mL Krebs-Ringer bicarbonate (KRB) buffer. (C) Digestion with trypsin potentiated the membrane-induced increase in [Ca²⁺]ᵢ. The membrane (100 μg/0.1 mL PBS) was incubated with trypsin (6 μg) at 37° C. for 30 minutes before it was applied to tracheal cells in 0.9 mL KRB. (D) The soluble membrane protein showed greater activity than undigested membrane in (A). The soluble protein was prepared by subjecting the trypsinized membrane to ultracentrifigation (100,000× g, 60 minutes) to obtain the supernatant.

FIG. 7 is a photograph of an immunoblot of Mhyo membrane polypeptides from pathogenic (P) and nonpathogenic (N) Mhyo probed with swine anti-Mhyo serum (1:80). Marker lane identified by the apparent molecular weight in kDa (10 μg/lane). Arrows denote the polypeptide bands observed in pathogenic, but not nonpathogenic Mhyo.

FIG. 8 is a photograph of an immunoblot of Mhyo membrane polypeptides from pathogenic (P) and nonpathogenic (N) Mhyo. The samples were digested with trypsin and probed with swine anti-Mhyo serum (1:80). Marker lane identified by the apparent molecular weight in kDa (10 μg/lane). Arrows denote the polypeptide bands observed in pathogenic, but not nonpathogenic Mhyo.

FIG. 9 is a graph plotting the purification of tryptic fragments of Mhyo membrane polypeptide using anion exchange HPLC. A linear gradient of 0-0.5 M NaCl in Tris buffer (pH 8.5) was used to elute the polypeptides. The elutes were monitored at an absorbance of 280 nm. The number 3 indicates fraction 3; while the number 4 indicates fraction 4.

FIG. 10 is a graph plotting fraction 4 (10 μg/mL)-induced [Ca²⁺]ᵢ increase in porcine ciliated tracheal cells (n=8 cells). This fraction evoked [Ca²⁺]ᵢ increase. The arrow indicates the administration of the polypeptide fraction.

FIG. 11 is a photograph of an immunoblot of Mhyo polypeptides probed with anti-Mhyo swine convalescent serum. The marker lane identifies the apparent molecule weight in kDa. Lane 1: fraction #4 (10 μg/lane); lane 2: soluble tryptic fragment of Mhyo before purification (10 μg/lane); lane 3: fraction #4 (from another purification run; 5 μg/lane); lane 4: Mhyo whole cell antigen (10 μg/lane); lane 5: blank (no antigen). The primary antibody was swine antiserum (1:100). The secondary antibody was goat anti-swine serum (1:1000). A positive band is observed at about 65 kDa in lanes 1 and 3.

FIG. 12 is a graph plotting the purification of tryptic fragments of Mhyo membrane protein using anion exchange HPLC. A linear gradient of 0-0.5 M NaCl in Tris buffer (pH 8.5) was used to elute the polypeptides. The elutes were monitored at an absorbance of 280 nm. Fraction 8 of the elutes exhibited Ca²⁺ releasing ability.

FIG. 13 is a graph plotting fraction 8 (1 μg)-induced [Ca²⁺]ᵢ, increase in porcine ciliated tracheal cells (n=4 cells). This fraction was the only eluted fraction that evoked [Ca²⁺]ᵢ increase. The arrow indicates the administration of the polypeptide fraction.

FIG. 14 is a graph plotting [Ca²⁺]ᵢ increase in porcine ciliated tracheal cells incubated with tryptic Mhyo membrane preparation pretreated with a soybean trypsin inhibitor (TI). TI failed to inhibit [Ca²⁺]ᵢ increase induced by the tryptic membrane preparation of Mhyo in ciliated tracheal epithelia. TI was incubated with the tryptic membrane preparation at 37° C. for 10 minutes prior to administration. Ordinate shows [Ca²⁺]ᵢ in nM. Each trace depicts [Ca²⁺]ᵢ changes in one cell.

### DETAILED DESCRIPTION

The invention provides methods and materials related to mycoplasma. For example, the invention provides mycoplasma polypeptides having the ability to increase calcium release from porcine ciliated tracheal cells as well as antibodies that bind to such mycoplasma polypeptides. In addition, the invention provides methods for identifying inhibitors of mycoplasma-induced calcium release from porcine ciliated tracheal cells.

In one embodiment, the invention provides substantially pure polypeptides. The term “polypeptide” as used herein refers to any chain of amino acid residues with or without one or more post-translational modifications (e.g., phosphorylation or glycosylation). The polypeptides provided herein can be any size. For example, a polypeptide having the ability to increase calcium release from porcine ciliated tracheal cells can be 10, 25, 50, 75, 100, 125, 150, 175, 200, or more amino acids in length. In addition, a polypeptide having the ability to increase calcium release from porcine ciliated tracheal cells can have a molecular weight that is between about 10 kDa and about 150 kDa. For example, a polypeptide having the ability to increase calcium release from porcine ciliated tracheal cells can have a molecular weight of about 10, 20, 30, 40, 50, 60, 65, 70, 75, 80, 85, 90, 95, 100, 105, 110, 115, or 120 kDa. In addition, a polypeptide having the ability to increase calcium release from cells (e.g., porcine ciliated tracheal cells) can be a tryptic fragment. In such cases, the molecular weight of the polypeptide following a tryptic digest can be between about 10 kDa and about 80 kDa. For example, the molecular weight of a polypeptide following a tryptic digest can be about 10, 15, 20, 25, 30, 35, 40, 45, 50, 55, 60, 65, 70, 75, or 80 kDa. In some embodiments, the polypeptide (e.g., full length polypeptide or tryptic fragment) having the ability to increase calcium release from cells (e.g., porcine ciliated tracheal cells) can be from a pathogenic Mhyo strain (e.g., pathogenic M. hyopneumoniae strain 91-3).

The term “amino acid residue” as used herein refers to natural amino acid residues, unnatural amino acid residues, and amino acid analogs, all in their D and L stereoisomers if their structures so allow. Natural amino acid residues include, without limitation, alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Unnatural amino acid residues include, without limitation, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine, pipecolic acid, and N-methylarginine.

The term “amino acid analog” as used herein refers to a compound that is structurally similar to a naturally occurring amino acid residue as is typically found in native polypeptides, but differs in composition such that either the C-terminal carboxy group, the N-terminal amino group, or the side-chain functional group has been chemically modified to another functional group. Amino acid analogs include, without limitation, aspartic acid-(beta-methyl ester), an analog of aspartic acid; N-ethylglycine, an analog of glycine; and alanine carboxamide, an analog of alanine. Other examples of amino acid residues and amino acid analogs are listed in Gross and Meienhofer, The Peptides: Analysis, Synthesis, Biology, Academic Press, Inc., New York (1983). Amino acid analogs can be naturally occurring or can be synthetically prepared.

Polypeptides can be modified for use in vivo by the addition, at the amino- or carboxy-terminal end, of a stabilizing agent to facilitate survival of the polypeptide in vivo. This can be useful in situations in which peptide termini tend to be degraded by proteases prior to cellular uptake. Such blocking agents can include, without limitation, additional related or unrelated amino acid sequences that can be attached to the amino- and/or carboxy-terminal residues of a polypeptide (e.g., an acetyl group attached to the N-terminal amino acid or an amide group attached to the C-terminal amino acid). Such attachment can be achieved either chemically, during the synthesis of the polypeptide, or by recombinant DNA technology using standard methods. Alternatively, blocking agents such as pyroglutamic acid or other molecules can be attached to the amino- and/or carboxy-terminal residues. In other embodiments, the amino group at the amino terminus and/or the carboxy group at the carboxy terminus can be replaced with a different moiety.

Polypeptides also can contain an amino acid tag. The term “amino acid tag” as used herein refers to a generally short amino acid sequence that provides a ready means of detection and/or purification through interactions with an antibody against the tag or through other compounds or molecules that recognize the tag. For example, amino acid tags such as c-myc, hemagglutinin, polyhistidine, or Flag® can be used to aid purification and detection of a polypeptide. As an example, a polypeptide with a polyhistidine tag can be purified based on the affinity of histidine residues for nickel ions (e.g., on a Ni—NTA column), and can be detected in western blots by an antibody against polyhistidine (e.g., the Penta-His antibody, Qiagen, Valencia, Calif.). Amino acid tags can be inserted anywhere within a polypeptide sequence. For example, an amino acid tag can be inserted at the amino- or carboxy-terminus of a polypeptide.

The polypeptides described herein can be obtained using any method. For example, a polypeptide having the ability to increase calcium release from cells can be obtained by extraction from a natural source (e.g., from Mhyo cells), by expression of a recombinant nucleic acid encoding the polypeptide, or by chemical synthesis (e.g., by solid-phase synthesis or other methods well known in the art, including synthesis with an ABI peptide synthesizer; Applied Biosystems, Foster City, Calif.). In addition, the polypeptides can be purified by, for example, high pressure liquid chromatography (e.g., reverse phase HPLC) or can be purified using gel electrophoresis. For example, the band corresponding to a particular polypeptide can be cut from a gel and eluted to obtain a polypeptide preparation.

The polypeptides provided herein can be substantially pure. The term “substantially pure” as used herein with reference to a polypeptide means the polypeptide is substantially free of other polypeptides, lipids, carbohydrates, and nucleic acid with which it is naturally associated. Thus, a substantially pure polypeptide is any polypeptide that is removed from its natural environment and is at least 60 percent free, preferably 75 percent free, and most preferably 90 percent free from other components with which it is naturally associated. The polypeptides provided herein can be 60, 65, 70, 75, 80, 85, 90, 95, or 99 percent pure. Typically, a substantially pure polypeptide will yield a single major band on a non-reducing polyacrylamide gel. It is understood that a Mhyo polypeptide is considered substantially pure if it has been purified and then mixed with, for example, an adjuvant or a pharmaceutical carrier, as the Mhyo polypeptide is separated from the cellular components with which it is associated in nature. Any method can be used to purify a polypeptide provided herein. For example, affinity chromatography, immunoprecipitation, size exclusion chromatography, and ion exchange chromatography can be used to purify a Mhyo polypeptide. The extent of purification can be measured by any appropriate method, including but not limited to: column chromatography, polyacrylamide gel electrophoresis, or high-performance liquid chromatography.

Any method can be used to determine whether a particular polypeptide increases calcium release from cells. For example, the techniques described herein can be used to measure calcium release from porcine ciliated tracheal cells.

The invention also provides antibodies that bind to the polypeptides provided herein. The term “antibody” as used herein refers to intact antibodies as well as antibody fragments that retain some ability to selectively bind an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have three dimensional structural characteristics as well as charge characteristics.

In one embodiment, the invention provides antibodies having specific binding affinity for a polypeptide provided herein. Such antibodies can be used in immunoassays in liquid phase or bound to a solid phase. For example, the antibodies provided herein can be used in competitive and non-competitive immunoassays in either a direct or indirect format. Examples of such immunoassays include the radioimmunoassay (RIA) and the sandwich (immunometric) assay.

The antibodies provided herein can be prepared using any method. For example, any substantially pure polypeptide provided herein, or fragment thereof, can be used as an immunogen to elicit an immune response in an animal such that specific antibodies are produced. Thus, an intact full-length polypeptide or fragments containing small peptides can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art (e.g., Green et al., Production of Polyclonal Antisera, In: Immunochemical Protocols (Manson, ed.), pages 1-5 (Humana Press 1992) and Coligan et al., Production of Polyclonal Antisera in Rabbits, Rats, Mice and Hamsters, In: Current Protocols i Immunology, section 2.4.1 (1992)). In addition, various techniques common in the immunology arts can be used to purify and/or concentrate polyclonal antibodies, as well as monoclonal antibodies (Coligan, et al., Unit 9, Current Protocols in Immunology, Wiley Interscience, 1994).

The preparation of monoclonal antibodies also is well-known to those skilled in the art (e.g., Kohler & Milstein, Nature 256:495 (1975); Coligan et al., sections 2.5.1-2.6.7; and Harlow et al., Antibodies: A Laboratory Manual, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include, without limitation, affinity chromatography with Protein-A Sepharose, size-exclusion chromatography, and ion-exchange chromatography (Coligan et al., sections 2.7.1-2.7.12 and sections 2.9.1-2.9.3; Barnes et al., Purification of Immunoglobulin G (IgG), In: Methods in Molecular Biology, Vol. 10, pages 79-104 (Humana Press 1992)).

In addition, methods of in vitro and in vivo multiplication of monoclonal antibodies are well-known to those skilled in the art. Multiplication in vitro can be carried out in suitable culture media such as Dulbecco's Modified Eagle Medium (MEM) or RPMI 1640 medium, optionally replenished by mammalian serum such as fetal calf serum, or trace elements and growth-sustaining supplements such as normal mouse peritoneal exudate cells, spleen cells, and bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg (U.S. Pat. Nos. 4,036,945 and 4,331,647) and others (Nisonhoff et al., Arch. Biochem. Biophys. 89:230 (1960); Porter, Biochem. J. 73:119 (1959); Edelman et al., Methods in Enzymology, Vol. 1, page 422 (Academic Press 1967); and Coligan et al. at sections 2.8.1-2.8.10 and 2.10.1-2.10.4).

In addition, the invention provides methods and materials that can be used to identify compounds that inhibit mycoplasma-induced calcium release (e.g., calcium release induced by Mhyo polypeptides) from cells (e.g., porcine ciliated tracheal cells). A method of identifying an inhibitor of mycoplasma-induced calcium release from cells can involve incubating cells (e.g., porcine ciliated tracheal cells) with a preparation containing a mycoplasma polypeptide (e.g., a Mhyo polypeptide from pathogenic Mhyo) in the presence of a test compound, and determining whether the test compound inhibits the cells from releasing calcium. In another embodiment, a method for identifying an inhibitor of calcium release can involve contacting cells with a mycoplasma polypeptide preparation pretreated with a test compound, and determining whether the test compound inhibits the cells from releasing calcium. Calcium release can be measured using any of the methods described herein. The preparation can be a crude Mhyo membrane polypeptide preparation, a purified Mhyo polypeptide preparation, or a tryptic digest of a Mhyo membrane polypeptide preparation. A test compound can be identified as an inhibitor of mycoplasma-induced calcium release if the increase in calcium release induced by the preparation containing the mycoplasma polypeptide is reduced in the presence of the compound as compared to in the absence of the compound. By “reduced” is meant that the occurrence of calcium release is lower (e.g., 5%, 10%, 25%, 50%, 75%, or 100% lower) in the presence of the test compound than in the absence of the compound. Any compound can be used as a test compound. For example, molecules that are polypeptides (e.g., proteases, antibodies, 10-50 amino acid polypeptides), oligonucleotides, esters, lipids, esters, carbohydrates, or steroids can be used as test compounds. Those of ordinary skill in the art can readily establish suitable amounts of test compounds and suitable incubation times.

The invention will be further described in the following examples, which do not limit the scope of the invention described in the claims.

### EXAMPLES

### Example 1

### Mycoplasma Hyopneumoniae Increases Intracellular Calcium Release in Porcine Ciliated Tracheal Cells

The effects of intact pathogenic Mycoplasma hyopneumoniae, nonpathogenic M. hyopneumoniae, and M. flocculare on intracellular free Ca²⁺ concentrations ([Ca²⁺]ᵢ) in porcine ciliated tracheal epithelial cells were determined. Briefly, the ciliated epithelial cells had basal [Ca²⁺]ᵢ of 103±3 nM (n=217 cells). The [Ca²⁺]ᵢ increased by 250±19 nM (n=47 cells) from the basal level within 100 seconds of addition of pathogenic M. hyopneumoniae strain 91-3 (300 μg/mL), which lasted about 60 seconds. In contrast, nonpathogenic M. hyopneumoniae and M. flocculare at 300 μg/mL failed to increase [Ca²⁺]ᵢ. In Ca²⁺-free medium, pathogenic M. hyopneumoniae still increased [Ca²⁺]ᵢ in tracheal cells. Pretreatment with thapsigargin (1 μM, 30 minutes), which depleted Ca²⁺ store in the endoplasmic reticulum, abolished the effect of M. hyopneumoniae. Pretreatment with pertussis toxin (100 ng/mL, 3 hours) or U-73122 (2 μM, 100 seconds), an inhibitor of phospholipase C, also abolished the effect of M. hyopneumoniae. The administration of Mastoparan 7, an activator of pertussis toxin-sensitive-protein (G_{i/o}), increased [Ca²⁺]ᵢ in ciliated tracheal cells. These results suggest that pathogenic M. hyopneumoniae activates receptors that are coupled to G_{i/o}, which in turn activates a phospholipase C pathway, thereby releasing Ca²⁺ from the endoplasmic reticulum. Thus, Ca²⁺ serves as a signal for the pathogenesis of M. hyopneunmoniae.

Methods and Materials

All reagents were obtained from Sigma Chemical (St. Louis, Mo.), except that fura-2 AM was obtained from Molecular Probes (Eugene, Oreg.) and U-73122, U-73343, and Mastoparan 7 (Mas 7) were obtained from Biomol (Plymouth Meeting, Pa.).

The following intact mycoplasmas were used herein: (1) a pathogenic M. hyopneumoniae strain 91-3, originally cloned from strain 232, which exhibits high adherence to cilia in a microtiter adherence assay (Zhang et al., Infect. Immun., 62:1616-1622 (1994)); (2) a nonpathogenic M. hyopneumoniae strain J (ATCC strain 25934), which does not adhere to cilia (Zielinski and Ross, Ant. J. Vet. Res., 54:1262-1269 (1993)); and M. flocculare strain Ms42 (ATCC strain 27399), which is nonpathogenic in swine. Mycoplasmas were cultured in Friis medium (Friis, Nord. Vet. Med., 27:337-339 (1975)) to logarithmic phase and harvested by centrifugation at 15,000× g for 30 minutes. Following centrifugation, the mycoplasma pellets were collected and washed three times with 50 mL of PBS by centrifugation at 15,000× g for 15 minutes. The final pellets were dispersed through a 27-gauge needle in PBS. The number of mycoplasma whole cells collected from 200 mL of culture (3.4±1.7×10¹¹ CCU, n=7) was determined as color changing units (CCU) using serial dilutions with tubes containing Friis medium. This cell density corresponded with 2.70±0.08 mg protein measured by the bicinchoninic acid method (Pierce, Rockford, Ill.) as previously described (Zhang et al., Infect. Immun., 62:4367-4373 (1994) and Zhang et al., Infect. Immun. 63:1013-1019 (1995)). The final mycoplasma concentration was adjusted to 3 mg protein/mL in PBS.

Tracheal cells were isolated as previously described (Young et al., Vet. Microbiol., 71:269-279 (1999)). Briefly, the tracheas were removed from 3-6 month old specific-pathogen-free pigs anesthetized with sodium pentobarbital using aseptic techniques. The ciliated cells were dissociated using 0.15% pronase and 0.01% DNase in Ca²⁺- and Mg²⁺-free MEM medium, which was incubated at 4° C. for 24 hours. The epithelial cells were collected by centrifugation at 125× g for 5 minutes. The cell pellets were resuspended in a mixture of DMEM (high glucose) and Ham's F-12 (1:1) media containing 5% FBS, 0.12 U/mL of insulin, and 100 U/mL of penicillin-streptomycin. Cell suspensions were transferred to 90-mm tissue culture dishes and incubated in 5% CO₂ for 60-90 minutes to remove fibroblasts. The tracheal epithelial cells were stored in liquid nitrogen until use.

The following techniques were used to obtain [Ca²⁺]ᵢ measurements in single cells. The tracheal cells were loaded with 4 μM fura-2 acetoxymethyl ester (fura-2AM) in Krebs-Ringer bicarbonate (KRB) buffer solution containing: 136 mM NaCl, 4.8 mM KCl, 1.5 mM CaCl₂, 1.2 mM KH₂PO₄, 1.2 mM MgSO₄, 10 mM HEPES, 5.5 mM glucose, and 0.1% BSA, pH 7.4 and incubated for 30 minutes at 37° C. The loaded cells were centrifuged (700× g, 2 minutes), then resuspended with KRB at a concentration of 500-1000 cells/mL. The tracheal cells loaded with fura-2AM were plated onto poly-lysine-coated coverslips in a custom-made Petri dish. The dish containing fura-2 loaded cells was mounted on the stage of an inverted fluorescence microscope (Carl Zeiss, NY). Only viable ciliated tracheal cells were focused on for the determination of [Ca²⁺]ᵢ at 24° C. The fura-2 loaded porcine ciliated tracheal cells deteriorated quickly at 37° C. Fluorescence images were obtained (excitation wavelengths of 334 and 380 nm; emission wavelength of 510±20 nm) and used to generate spatially resolved maps of [Ca²⁺]ᵢ by subtracting the background dividing the images on a pixel-by-pixel basis. The emitted signals were digitalized, recorded, and processed using the Attofluor digital fluorescence imaging system (Atto Instruments, Rockville, Md.). After reading fluorescence for 150 seconds, mycoplasmas were mixed with the cell system. [Ca²⁺]ᵢ was calculated as previously described (Grynkiewicz et al., J. Biol. Chem., 260:3440-3450 (1985)). Calibration was performed in situ according to the procedure provided by Atto Instruments, using Fura-2 penta K⁺ as a standard.

To compare the [Ca²⁺]ᵢ of tracheal cells response to pathogenic M. hyopneumoniae strain 91-3, avirulent M. hyopneumoniae, and M. flocculare, the cells were treated with the same concentration of 300 μg/mL. One to five ciliated single tracheal cells in each experiment were selected to investigate the [Ca²⁺]ᵢ changes. The mycoplasmas were maintained on ice before being applied to tracheal cells.

To investigate the pathway of Ca²⁺ signaling, pertussis toxin (PTX, 100 ng/mL) was preincubated with tracheal cells for 3 hours. Cells were pretreated with thapsigargin (TG, 1 μM) for 30 minutes at 37° C. prior to the addition of the mycoplasmas to deplete the ER Ca²⁺ store (Thastrup et al., Proc. Natl. Acad. Sci. USA, 87:2466-2470 (1990)). Cells were pretreated with U-73122 (2 μM), a phospholipase C inhibitor (Bleasdale and Fisher, Neuroprotocols, 3:125-133 (1993)) or its inactive analogue U-73343 for 100 seconds at 37° C. prior to the addition of the mycoplasmas. To confirm that mycoplasmas increased [Ca²⁺]ᵢ by activating a G_{i/o} protein, Mas 7 (10 μM), an activator of this protein (Higashijima et al., J. Biol. Chem., 265:14176-14186 (1990)), was used to determine if it can increase [Ca²⁺]ᵢ in tracheal cells. In addition, it was determined whether PTX can block the increase in [Ca²⁺]ᵢ due to Mas 7.

Data on [Ca²⁺]ᵢ were analyzed by ANOVA or by Student's t-test. The significance level was set at P<0.05.

Effects of Mycoplasmas on [Ca^{2+]}ᵢ in Porcine Ciliated Tracheal Epithelial Cells

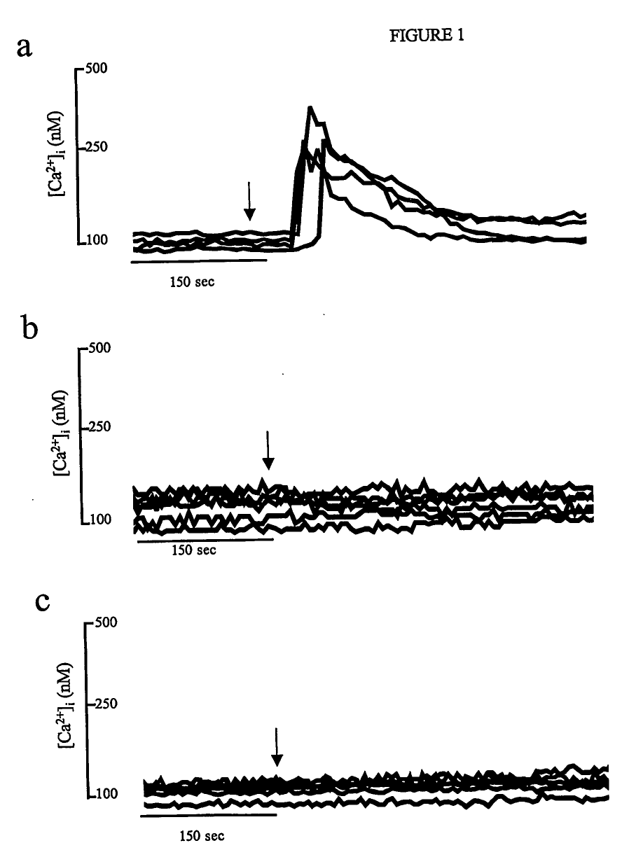

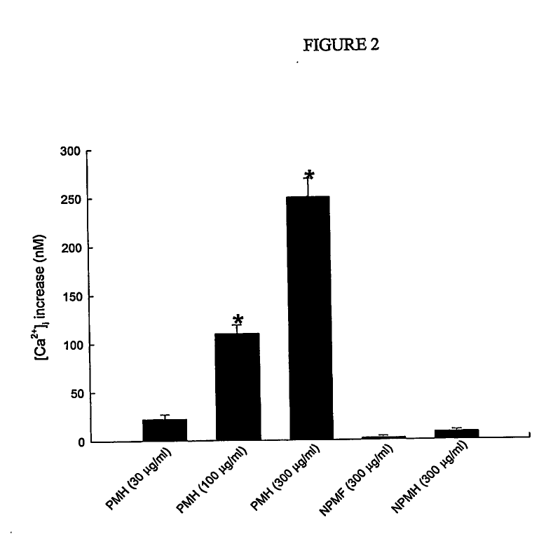

M. hyopneumoniae strain 91-3 binds to cilia of porcine tracheal cells (Debey et al., Am. J. Vet. Res., 53:1705-1710 (1992); Mebus and Underdahl, Am. J. Vet. Res. 38:1249-1254 (1977); and Tajima and Yagihashi, Infect. Immun., 37:1162-1169 (1982)). The changes in [Ca²⁺]ᵢ were determined after the inoculation of ciliated tracheal cells with strain 91-3. The ciliated epithelial cells had basal [Ca^{2+]}ᵢ of 103±3 nM (n=217 cells). After being exposed to M. hyopneumoniae strain 91-3 at 300 μg/mL, an increase in [Ca²⁺]ᵢ in 89 percent (47 of 53 cells in 10 experiments) of the cells was observed. As shown in FIGS. 1 and 2, administration of pathogenic M. hyopneumoniae strain 91-3 (300 μg/mL) increased [Ca²⁺]ᵢ in ciliated cells within 100 seconds. In contrast, nonpathogenic M. hyopneumoniae (18 cells in 6 experiments) and M. flocculare (24 cells in 8 experiments) did not increase [Ca²⁺]ᵢ at the same mycoplasma concentration (300 μg/mL) (FIG. 1).

In a dose-response study, 30 μg/mL of M. hyopneumoniae strain 91-3 (18 cells in 6 experiments) did not significantly change [Ca²⁺]ᵢ (FIG. 2). However, 100 μg/mL (16 cells in 7 experiments; 84 percent of cells responded) and 300 μg/mL (47 cells in 10 experiments; 89 percent of cells responded) increased [Ca²⁺]ᵢ by 110±9 nM and 250±19 nM, respectively (FIG. 2).

Since M. hyopneumoniae strain 91-3 might increase [Ca²⁺]ᵢ in ciliated cells via its secretory product, supernatants were collected from the mycoplasma (300 μg/mL) following the centrifugation at 15,000× g for 15 minutes to test its ability in increasing [Ca²⁺]ᵢ. These supernatants did not increase [Ca²⁺]ᵢ in ciliated cells.

Effects of M. hyopneumoniae Strain 91-3 in Ca²⁺-Free Medium

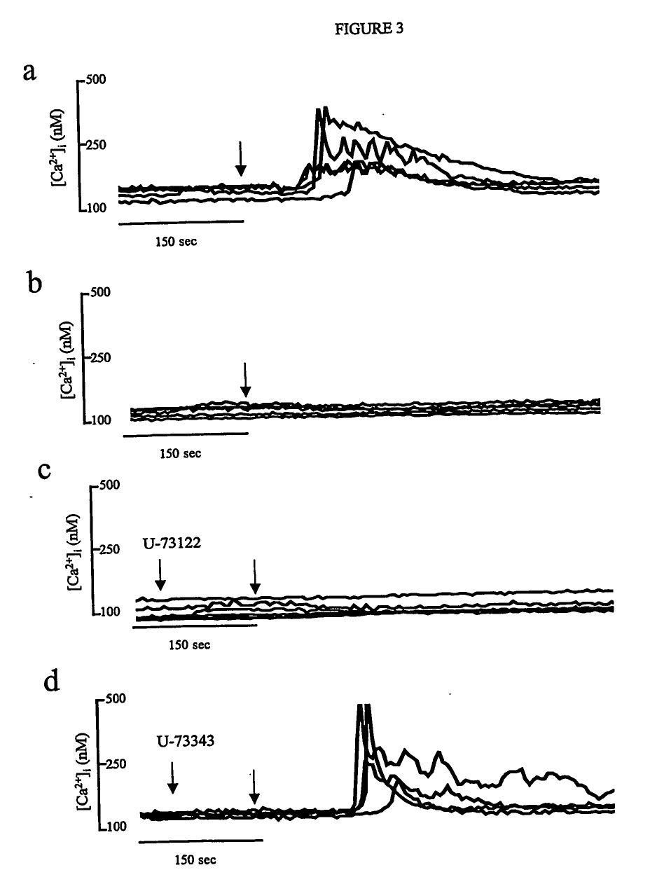

To determine the involvement of extracellular Ca²⁺, experiments were performed using Ca²⁺-free medium supplemented with 10 μM EGTA, a Ca²⁺ chelator. M. hyopneumoniae strain 91-3 (300 μg/mL) still increased [Ca²⁺]ᵢ (before: 117±6 nM, after: 324±31 nM, 10 cells in 4 experiments; 84 percent of cells responded) (FIG. 3a). These results indicate that the increase is attributable to Ca²⁺ release from intracellular stores, rather than through a Ca²⁺ influx mechanism.

Effect of TG on M. hyopneumoniae-Induced [Ca²⁺]ᵢ Increase

To determine whether the endoplasmic reticulum (ER) was the source of Ca²⁺ release, ciliated cells were treated with 1 μM TG, a microsomal Ca²⁺-ATPase inhibitor, for 30 minutes. In previous studies, TG was found to deplete the ER Ca²⁺ store (Thastrup et al., Proc. Natl. Acad. Sci. USA, 87:2466-2470 (1990)), since it abolished ionomycin-induced intracellular Ca²⁺ release from porcine ciliated tracheal cells. Similarly, TG treatment abolished M. hyopneumoniae strain 91-3 (300 μg/mL)-induced [Ca²⁺]ᵢ increase, demonstrating that this organism evokes ER Ca²⁺ release from porcine tracheal epithelial cells. (FIG. 3b).

Effects of U-73122 and U-73433 on M. hyopneumoniae-Induced [Ca²⁺]ᵢ increase

Since inositol 1,4,5-trisphosphate (IP₃) releases Ca²⁺ from the ER, and IP₃ production is catalyzed by phospholipase C (PLC), the following experiment was performed. Pretreatment of tracheal cells with 2 μM U-73122, a specific PLC inhibitor (Bleasdale and Fisher, Neuroprotocols, 3:125-133 (1993)), before inoculation with M. hyopneumoniae strain 91-3 abolished the mycoplasma-induced [Ca²⁺]ᵢ increase in the ciliated cells (FIG. 3c). In contrast, U-73343, an inactive analogue of U-73122, did not prevent the [Ca²⁺]ᵢ response to the mycoplasma (basal: 90±12 nM, peak: 330±25 nM, 10 cells in 4 experiments; 82 percent of cells responded) (FIG. 3d). These findings support that the [Ca²⁺]ᵢ increase by M. hyopneumoniae is mediated by activation of PLC.

Effects of PTX on M. hyopneunmoniae and Mas 7-Induced [Ca²⁺]ᵢ Increase

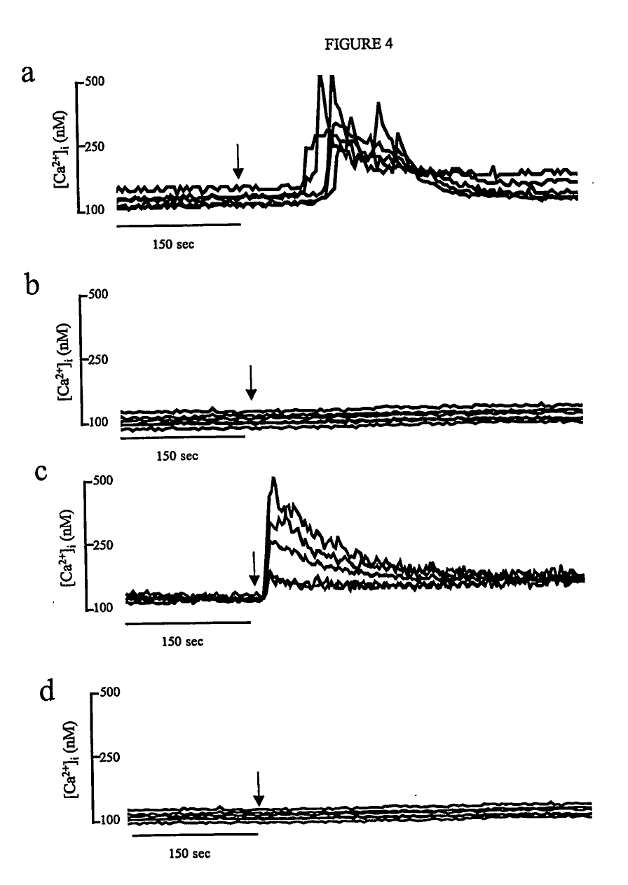

The following experiments were performed to assess whether a PTX-sensitive G protein mediates the effect of M. hyopneumoniae strain 91-3. In untreated control cells, M. hyopneumoniae strain 91-3 increased [Ca²⁺]ᵢ (254±57 nM, 9 cells in 3 experiments; 81 percent of cells responded; FIG. 4a). In contrast, pretreatment of ciliated cells with 100 ng PTX/mL for 3 hours abolished M. hyopneumoniae-induced increases in [Ca²⁺]ᵢ (FIG. 4b). These results indicate that M. hyopneumoniae activates receptors that are coupled to a PTX-sensitive G-protein (G_{i/o}). To confirm that G_{i/o} proteins are involved in the [Ca²⁺]ᵢ increase in the tracheal cells, the effect of Mas 7, an activator of G_{i/o} (Higashijima et al., J. Biol. Chem., 265:14176-14186 (1990)), on [Ca²⁺]ᵢ was studied. Administration of 10 μM Mas 7 to ciliated tracheal cells evoked an increase in [Ca²⁺]ᵢ from the basal level of 103±4 nM to 351±24 nM (n=9 cells in 3 experiments, 82 percent of cells responded) within 100 seconds (FIG. 4c). Pretreatment of these cells with PTX abolished the effect of Mas 7 (FIG. 4d). These results demonstrate that activation of G_{1/o} in ciliated tracheal cells increases [Ca²⁺]ᵢ.

M. hyopneumoniae colonizes the swine respiratory tract by binding to ciliated epithelial cells (Mebus and Underdahl, Am. J. Vet. Res., 38:1249-1254 (1977); Tajima and Yagihashi, Infect. Immun., 37:1162-1169 (1982); and Zhang et al., Infect. Inmmun., 62:1616-1622 (1994)). Adherence is mediated through a surface protein P97 (Hsu and Minion, Infect. Immun., 66:4762-4766 (1998); Hsu et al., J. Bacteriol., 179:1317-1323 (1997); and Minion et al., Infect. Immun., 68:3056-3060 (2000)). Ciliostasis and cilia loss quickly ensues (Debey and Ross, Infect. Inmmun., 62:5312-5318 (1994)). As demonstrated herein, Ca²⁺ flux is linked to cilia loss. Pathogenic M. hyopneumoniae strain 91-3 increased [Ca²⁺]ᵢ in porcine ciliated tracheal cells. In contrast, the nonpathogenic strain J of M. hyopneumoniae and M. flocculare failed to do so, indicating that binding to cilia was a prerequisite for Ca²⁺ flux induction. M. hyopneumoniae strain J does not bind to swine cilia (Zhang et al., Infect. Immun., 63:1013-1019 (1995)). The [Ca²⁺]ᵢ response was a rapid event, and the increase was dependent on mycoplasma concentration. In another study of [Ca²⁺]ᵢ increase by M. hyopneumoniae in neutrophils, 10⁷-10¹⁰ CCU of the pathogenic strain enhanced zymosan-induced increase in [Ca²⁺]ᵢ, whereas nonpathogenic strain did not (Debey et al., Vet. Res. Commun., 17:249-257 (1993)). Adherence of pathogenic M. hyopneumoniae strain 91-3 (10⁹ CCU) to the cilia of respiratory epithelia results in tangling, clumping, and longitudinal splitting within 90 minutes of the mycoplasma administration, whereas nonpathogenic M. hyopneumoniae strain does not show ciliary damages (Debey et al., Am. J. Vet. Res., 53:1705-1710 (1992) and Young et al., Vet. Microbiol., 71:269-279 (1999)). Thus, changes in [Ca²⁺]ᵢ in the tracheal epithelia is involved in the pathogenesis of M. hyopneumoniae.

The magnitude of the [Ca²⁺]ᵢ increase in isolated ciliated cells in response to M. hyopneumoniae varied from cell to cell, but in general, it increased with increasing concentration of mycoplasma. This heterogeneity of Ca²⁺ response in the airway epithelial cells was similar to the effect of extracellular ATP reported in glial cells (VandenPol et al., J. Neurosci., 12:2648-2664 (1992)), bile duct cells (Nathanson et al., Am. J. Physiol., 271:G86-G96 (1996)), megakaryocytes (Tertyshnikova and Fein, Cell Calcium, 21:331-344 (1997)), and chondrocytes (D'Andrea and Vittur, J. Bone Miner. Res., 11:946-954 (1996)). In respiratory epithelial cells of rabbits, the heterogeneity of Ca²⁺ response is due to the sensitivity of individual cells to extracellular ATP (Evens and Sanderson, Am. J. Physiol., 277:L30-L41 (1999) and Korngreen et al., J. Physiol. (Lond.) 508:703-720 (1998)).

Increased [Ca²⁺]ᵢ due to microorganisms or their toxins has been reported in other bacteria. Intact S. typhimurium increases [Ca²⁺]ᵢ in intestinal epithelia, which mediates the increase in IL-8 secretion from these cells (Gewirtz et al., J. Clin. Invest., 105:79-92 (2000) and Pace et al., Cell, 72:505-514 (1993)). How S. typhimurium induces an increase in [Ca²⁺]ᵢ is not yet clear. E. coli enterotoxin elevates [Ca²⁺]ᵢ by releasing ER Ca²⁺]from HEp-2 cells (Baldwin et al., Infect. Immun., 59:1599-1604 (1991)). This release is attributable to activation of ryanodine receptor Ca²⁺ release channels, since the effect is blocked by a ryanodine receptor antagonist dantrolene (Danko et al., Biochim. Biophys. Acta., 816:18-24 (1985) and Heine and Wicher, Neuroreport, 9:3309-3314 (1998)). Intact verocytotoxin-producing E. coli, however, release Ca²⁺ from HEp-2 cells via the IP₃ pathway (Ismaili et al., Infect. Immun., 63:3316-3326 (1995)). Pyocyanine, an oxidant virulent factor secreted by Pseudomonas aeruginosa, increases IP₃ formation and [Ca²⁺]ᵢ in human airway epithelial cells, but reduces G-protein coupled receptor agonist-induced increase in IP₃ and [Ca²⁺]ᵢ (Denning et al., Am. J. Physiol., 274:L893-L900 (1998)). Pyocyanine-induced oxidation may be responsible for the increase in IP₃ formation (Denning et al., Am. J. Physiol., 274:L893-L900 (1998)). Pasteurella multocida toxin (PMT) increases [Ca²⁺]ᵢ in different intact animal cells by activating G_{q}-coupled PLC-β₁ isozyme (Wilson et al., J. Biol. Chem., 272:1268-1275 (1997)). This effect of PMT is largely attributable to its direct activation of G_{q}-PLC pathway, since microinjection of PMT into Xenopus oocytes, which bypasses the plasma membrane receptors, still activates G_{q}-PLC (Wilson et al., J. Biol. Chem., 272:1268-1275 (1997)).

Some extracellular bacterial structures can increase [Ca²⁺]ᵢ of host cells. For example, Type IV pili of pathogenic Neisseria adhere to an epithelial-like human cell line ME180 derived from cervical carcinoma, and increase [Ca²⁺]ᵢ via the pilus receptors (Kallstrom et al., J. Biol. Chem., 273:21777-217782 (1998)). Elevation of [Ca²⁺]ᵢ is needed as an initial step to establish a stable contact between the bacteria and host cells (Kallstrom et al., J. Biol. Chem., 273:21777-217782 (1998)). However, it is not clear how the pili of Neisseria cause an increase in [Ca²⁺]ᵢ.

Pathogenic M. hyopneumoniae strain 91-3 increased [Ca²⁺]ᵢ in the ciliated cells in Ca²⁺-free medium, indicating that the increase in [Ca²⁺]ᵢ is attributable to Ca²⁺ release from intracellular stores. Pretreatment of tracheal cells with TG to deplete ER Ca²⁺ store abolished the effect of the mycoplasma, confirming the involvement of this organelle in the Ca²⁺ release. Pretreatment of tracheal cells with U-73122, a specific PLC inhibitor, also prevented the mycoplasma-induced [Ca²⁺]ᵢ increase, indicating that the mycoplasma-induced Ca²⁺ release from the ER is via a PLC pathway.

The results provided herein indicate that receptors of M. hyopneumoniae in respiratory epithelium are coupled to G_{i/o}. Activating PLC is also consistent with observations with A₁ adenosine receptor-mediated phenomenon (Tomura et al., J. Biol. Chem., 272:23130-23137 (1997)). G_{i/o} proteins are usually responsible for the inhibition of adenylyl cyclase, regulation of K⁺ and Ca²⁺ channels, and activation of cGMP phosphodiesterase. Among G_{i/o} proteins, Gᵢ₂ and Gᵢ₃ can mediate the modulation of two signaling pathways; activation of PLC is mediated by G_{βy} dimer, whereas inhibition of adenylyl cylase is mediated by αᵢ (Tomura et al., J. Biol. Chem., 272:23130-23137 (1997)).

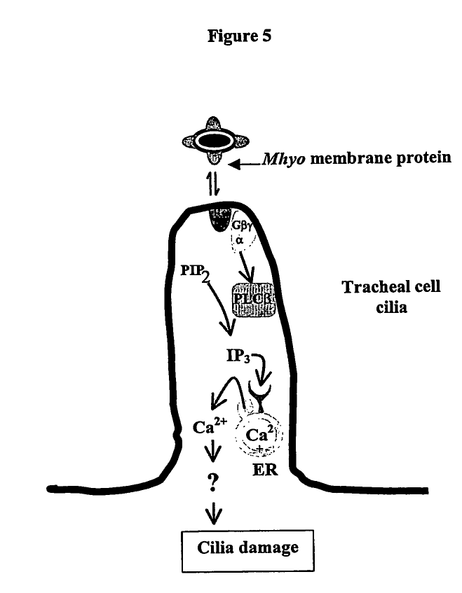

In summary, the results provided herein indicate that the receptors for pathogenic M. hyopneumoniae are coupled to G_{i/o.} Once binding of these receptors has occurred, this G protein stimulates the PLC pathway to increase [Ca2+]ᵢ through a rise in Ca²⁺ release from the ER (FIG. 5). In addition, experiments with adhesins demonstrated that adhesins from M. hyopneumoniae including P97 failed to increase [Ca²⁺]ᵢ. Also, inoculation of porcine ciliated tracheal cells with M. hyopneumoniae strain 91-3 increased ciliary beating frequency within 3 minutes of inoculation, which corresponded with the increase in [Ca²⁺]ₒ in these cells. These results were consistent with what have been found with Ca²⁺ action on ciliary beating frequency in ovine airway epithelial cells (Salathe and Bookman, J. Physiol. (Lond.), 520:851-865 (1999)), and support the involvement of changes in [Ca¹⁺]ᵢ in the pathogenesis of mycoplasma.

### Example 2

### Characterization and Purification of Mhyo Polypeptide that Induces [Ca²⁺]ᵢ Increases in Porcine Ciliated Tracheal Cells

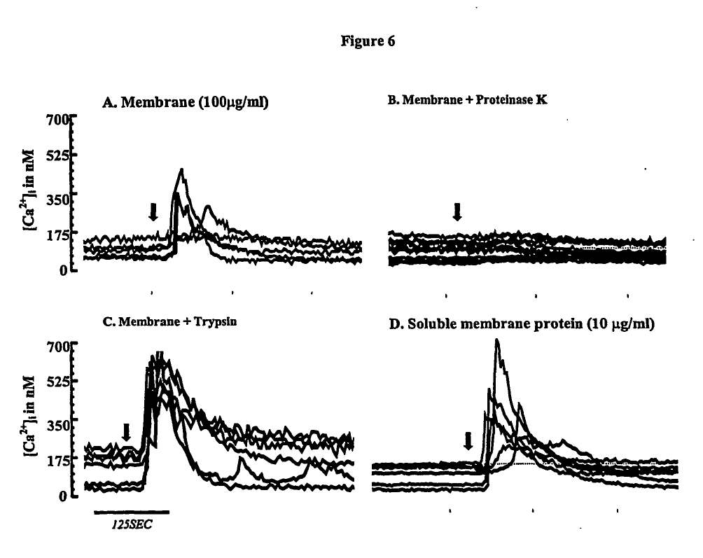

Mycoplasmas lack cell walls and have only one type of membrane, the plasma membrane (Razin S. (1993) Mycoplasma membranes as models in membrane research (Chapter 2), In: Subcellular Biochemistry. Vol 20: Mycoplasma Cell Membranes, edited by Rottem S, Kahane I. Plenum Press, New York. pp. 1-28). The Mhyo membrane was prepared by osmotic lysis of the organisms and tested to determine if it increased [Ca²⁺]ᵢ in ciliated tracheal cells. The Mhyo membrane increased [Ca²⁺ᵢ in ciliated tracheal cells (FIG. 6A). Pretreatment of the membrane with a proteolytic enzyme proteinase K or papain for 8 hours abolished the effect of the membrane (FIG. 6B). These results demonstrate that a membrane polypeptide is responsible for this effect. Interestingly, pretreatment with trypsin for 30 minutes, not only failed to reduce the [Ca²⁺]ᵢ increase, but even potentiated the effect of the membrane (FIG. 6C). Pretreatment with trypsin for 16 hours still failed to reduce the effect of the membrane on [Ca²⁺]₁. The trypsinization of the membrane can yield polypeptide fragments containing more epitopes for the receptors. The tryptic fragments of the mycoplasma were subjected to ultracentrifugation (100,000× g, 60 minutes). The resulting supernatant, which contains soluble polypeptides, also increased [Ca²⁺]ᵢ in ciliated epithelia. The [Ca²⁺]ᵢ elevating activity of this solubilized membrane polypeptide was at least 10 times more potent than the undigested membrane (FIG. 6D).

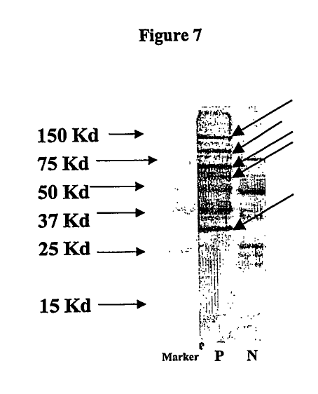

A western blot technique was used to compare outer membrane polypeptides from pathogenic Mhyo (91-3) and nonpathogenic Mhyo (strain J). The sample from pathogenic Mhyo exhibited five polypeptide bands not exhibited in the sample from nonpathogenic Mhyo (FIG. 7). The five polypeptide bands corresponded to molecular weights 30, 60, 65, 90, and 120 kDa, respectively.

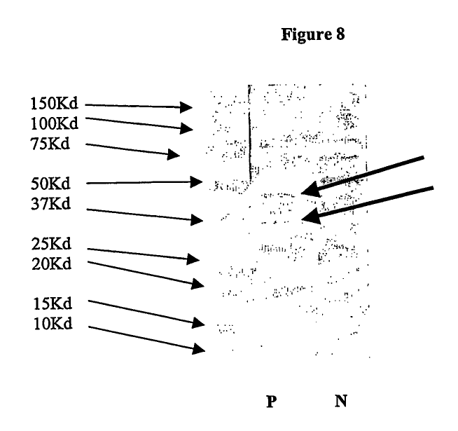

A western blot technique was used to compare outer membrane polypeptides from pathogenic Mhyo (91-3) and nonpathogenic Mhyo (strain J) after digestion with trypsin. The sample from pathogenic Mhyo exhibited two polypeptide bands not exhibited in the sample from nonpathogenic Mhyo (FIG. 8). The two polypeptide bands corresponded to molecular weights 35 and 50 kDa, respectively.

Gel electrophoresis (21 cm×50 cm) is used to collect these five polypeptides in quantities greater than about 10 μg. Once collected, the polypeptide preparation are used to perform [Ca²⁺]ᵢ assays to confirm which polypeptide increases [Ca²⁺]ᵢ in ciliated tracheal cells. In addition, 2-dimensional electrophoresis is used to further purify each polypeptide. Mass spectrometry is used to confirm the purity of each polypeptide prior to performing N-terminal protein sequencing. Once the N-terminal amino acid sequence is determined, sequence databases are searched to identify the amino acid sequence of the full length Mhyo polypeptide.

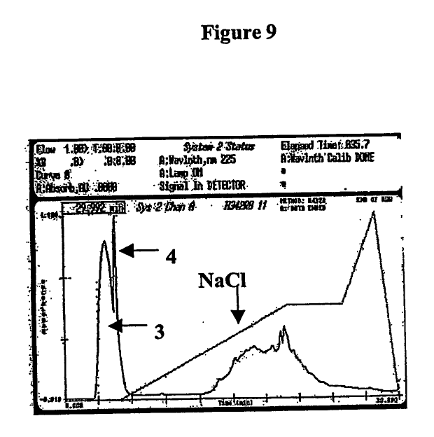

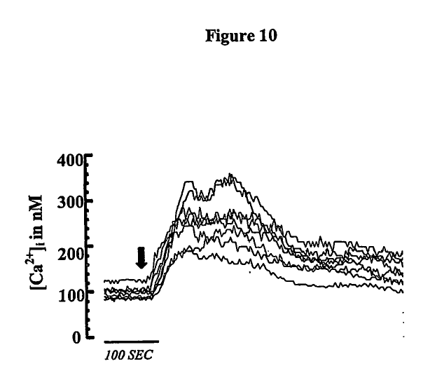

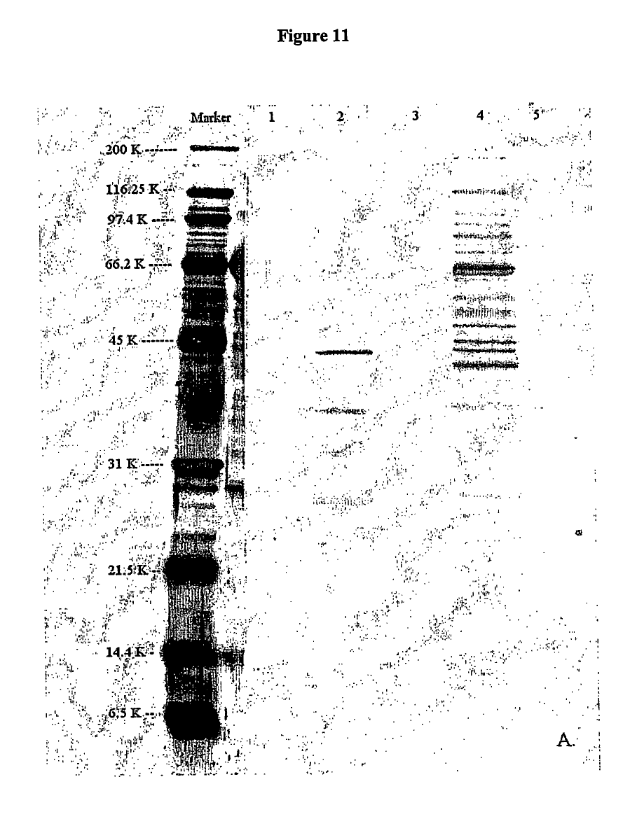

The solubilized Mhyo polypeptide was purified by HPLC using anion exchange column with a linear gradient of 0-0.5 M NaCl in Tris buffer (pH 8.5; FIG. 9). An early fraction, fraction #4, exhibited [Ca²⁺]ᵢ elevating activity in ciliated tracheal cells (FIG. 10). Western blot analysis revealed that fraction #4 contained a 65 kDa band that was recognized by anti-Mhyo convalescent serum (FIG. 11). This 65 kDa polypeptide band also appeared in the Mhyo whole cell preparation.

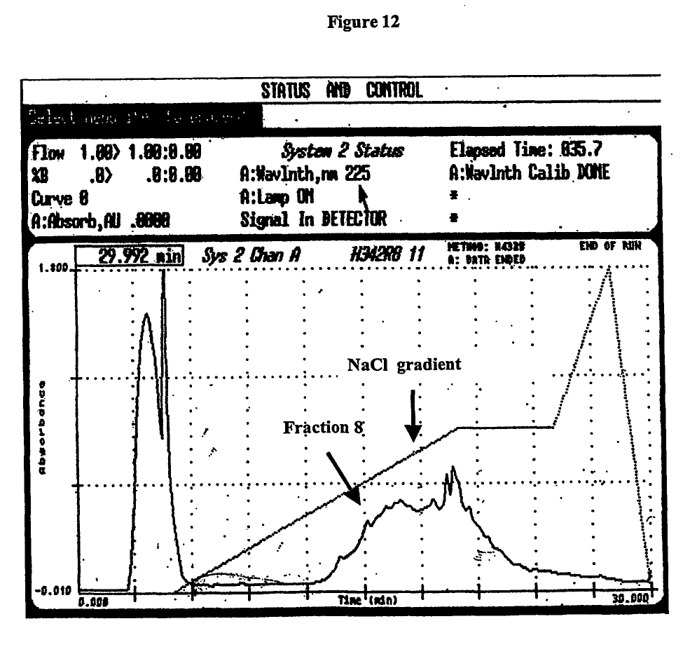

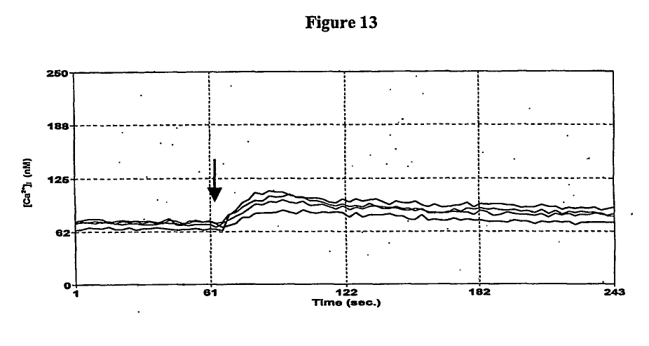

Since fraction #4 corresponded to a peak that eluted just before or slightly after application of the NaCl gradient, further analysis of later fractions was performed. This analysis revealed that fraction #8 increased [Ca²⁺]ᵢ in ciliated tracheal cells as well (FIGS. 12 and 13). Fraction #8 came off at the NaCl gradient of 0.4 M. These results indicated that fraction #8 contained a purified outer membrane Mhyo polypeptide that exhibits [Ca²⁺]ᵢ elevating activity in ciliated tracheal epithelia.

Using centrifugation filters, the size of the tryptic polypeptide fragment capable of [Ca²⁺]ᵢ increases in ciliated tracheal epithelia was determined. The filtrate following the use of a 30 kDa pore size filter failed to increase [Ca²⁺]ᵢ, while the filtrate following the use of a 100 kDa pore size filter increased [Ca²⁺]ᵢ in ciliated tracheal epithelia. These results indicate that the tryptic polypeptide fragment that is responsible for [Ca²⁺]ᵢ increases in ciliated tracheal epithelia can be between about 30 and about 100 kDa in size.

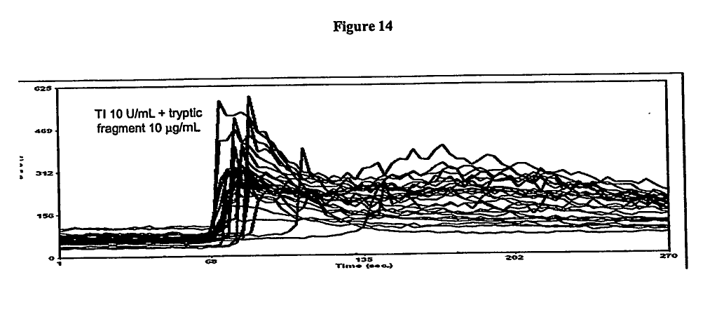

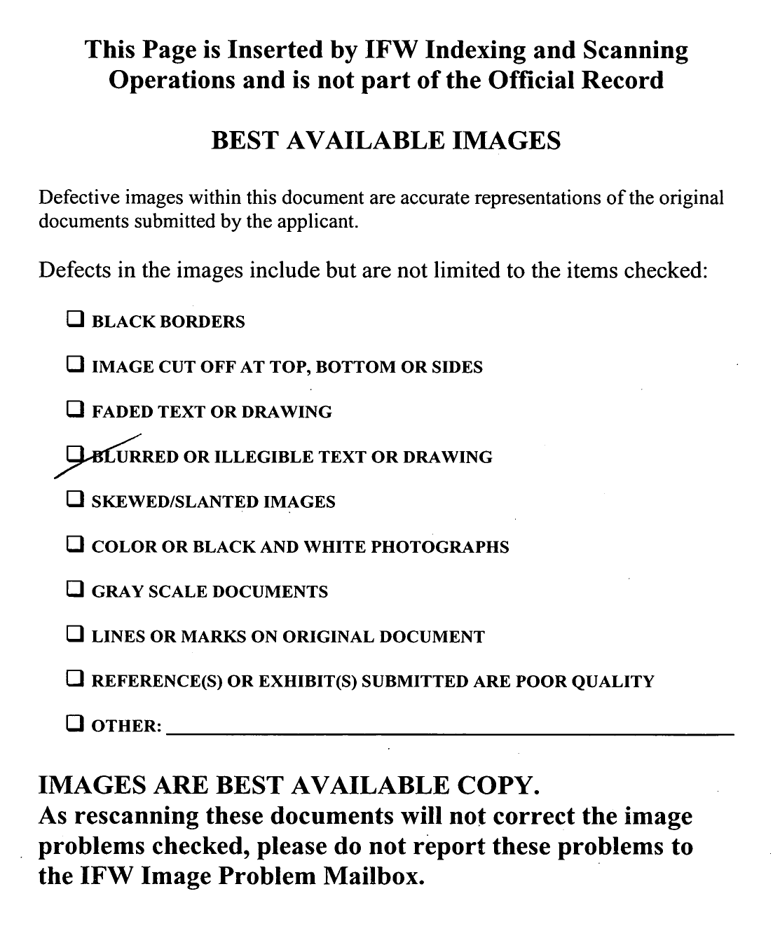

According to a recent report, trypsin at 0.1 U/mL can increase [Ca²⁺]ᵢ in guinea pig tracheal epithelia (Oshiro et al., Life Sci., 71:547-558 (2002)). Since the estimated trypsin concentration in the [Ca²⁺]ᵢ experiments described herein is about 1 U/mL, we tested whether trypsin plays a role in the observed tryptic fragment-induced [Ca²⁺]ᵢ increase. Trypsin alone at ≧1 U/mL was found to increase [Ca^{2+l ]}ᵢ in swine ciliated epithelia. Treatment with soybean trypsin inhibitor (10 U/mL), however, inhibited trypsin (10 U/mL)-induced [Ca²⁺]ᵢ increase, but failed to block the observed tryptic Mhyo polypeptide fragment-induced [Ca²⁺]ᵢ increase (FIG. 14). These results demonstrate that the stimulatory effect of the tryptic Mhyo preparation on [Ca²⁺]ᵢ is attributed to the Mhyo polypeptide, not trypsin.

### Example 3

### Obtaining Amino Acid Sequence of the Mhyo Polypeptide that Induces

[Ca²⁺]ᵢ Increases in Porcine Ciliated Tracheal Cells

The virulent Mhyo strain 91-3 is grown in Friis medium supplemented with 20% mycoplasma-free swine serum and harvested by centrifugation as previously described (Zhang et al., Infect Immun 62:1616-1622 (1994)). The organisms are subjected to osmotic lysis and centrifugation (35,000× g, 60 minutes) to obtain a membrane preparation as previously described (Pollack J D. (1998) Enzyme analysis (Chapter 10), In: Methods in Molecular Biology. Vol. 104: Mycoplasma Protocols, edited by Miles R & Nicholas A. Humana Press, Totowa, N.J. pp. 79-93). The membrane preparation is suspended in PBS and treated with trypsin at 17:1 ratio (w/w) at 37° C. for 30 minutes, followed by ultracentrifugation (100,000× g, 60 minutes). The resulting supernatant, which contains the active tryptic fragment, is purified by HPLC using an anion exchange column (Waters, Model DEA 5TW) and a linear gradient of 0-0.5 M NaCl in Tris buffer (pH 8.5). The elute is monitored at an absorbance of 280 nm, and fractions 4 and 8 are collected and further purified by C₁₈ reversed-phase HPLC using a linear gradient of 0-60% acetonitrile in 0.08% trifluoroacetic acid in water. Alternatively, gel filtration, hydrophobic interaction, or size-exclusion column techniques are used to further purify the polypeptide. The purified polypeptide is concentrated using a Sep-pak and eluted with acetonitrile-methanol as the solvent system. The solvent is removed under a stream of nitrogen. In addition, SDS-PAGE is used to confirm the molecular weight of the polypeptide. To follow purification, the elutes collected from noticeable peaks are tested for their ability to increase [Ca²⁺]ᵢ in ciliated tracheal cells. Purity of the resulting polypeptide preparation is determined by mass spectrometry (Voyager, Model DE PRO).

The polypeptide purified by C₁₈ HPLC and confirmed by mass spectrometry is subjected to N-terminal amino acid sequencing using an Applied Biosystems protein sequencer (Model 494). Alternatively, internal sequence information is obtained from fragments generated using cyanogen bromide cleavage or enzymatic cleavage such as by endoprotease Lys-C. The cleavage fragment are purified and subjected to N-terminal amino acid sequencing. Once the N-terminal amino acid sequence is determined, sequence databases are searched to identify the amino acid sequence of the full length Mhyo polypeptide.

The following methods are used to measure increased [Ca²⁺]ᵢ in ciliated tracheal cells. Tracheal cells are obtained from Mhyo-free pigs as described by Yamaya et al. (Am. J. Physiol., 262:L713-L724 (1992)). Briefly, the ciliated tracheal epithelial cells are isolated by enzyme digestion using 0.15% pronase and 0.01% DNase in Ca²⁺ and Mg²⁺-free MEM media and incubated at 4° C. for 24 hours. Enzyme digestion is stopped by the addition of fetal bovine serum. The cells are removed from the tracheas and washed by centrifugation in Dulbecco's MEM and Ham's F-12 (1:1) media. These cells are frozen in liquid nitrogen. When ready to be used, the cells are thawed quickly at 37° C. and allowed to attach to coverslips in the 5 mm-well of custom-made 30-mm Petri dishes coated with polysine in Krebs-Ringer bicarbonate buffer (KRB). The volume of incubation in such a dish is 200 μL. The [Ca²⁺]ᵢ determination procedure for single cells is performed using an image system as previously described (ZhuGe and Hsu, J. Pharmacol. Exp. Ther., 275:1077-1083 (1995)).

The [Ca²⁺]ᵢ data from a particular experiment are calculated by averaging the peaks of the increase in [Ca²⁺]ᵢ from at least 5 single cells in the same treatment group and compared to the control group, which receives a placebo (KRB). The Ca²⁺ bioassay is repeated once to confirm previous results. The data are analyzed using ANOVA, and mean comparisons are performed using Tukey's test. The a level is set at P≦0.05.

### Example 4

### Expressing and Characterizing Recombinant Mhyo Polypeptide that Induces [Ca²⁺]ᵢ Increases in Porcine Ciliated Tracheal Cells

A recombinant [Ca²⁺]ᵢ-elevating membrane Mhyo polypeptide (or fragments thereof) is obtained using methods similar to those described elsewhere (Hsu and Minion, Infect. Immun., 66:4762-4766 (1998)). Mycoplasmas use UGA, which is normally a stop codon, as a tryptophan coding codon. Thus, suppressor systems are used for expression of most mycoplasma gene sequences in E. coli. Alternatively, site directed mutagenesis is used to modify the UGA codons.

The nucleic acid encoding the Mhyo polypeptide (or fragment thereof) is cloned into a polyhistidine fusion expression vector such as pTrcHis to facilitate purification of the recombinant product. Recombinant E. coli is induced with IPTG, and the production of the recombinant Mhyo polypeptide is monitored by immunoblot using anti-polyhistidine. The induced E. coli are permeabilized with B—PER reagent (Pierce), and the cell debris is removed by centrifugation. The recombinant proteins is purified by metal chelate chromatography using either Talon (Clontech) or ProBond (Invitrogen) resins. The biological activity of the polypeptide is tested to confirm its ability in increasing [Ca²⁺]ᵢ in ciliated tracheal cells. For large batches, a Bio-Rad Biologic Chromatography system is used.

The recombinant Mhyo polypeptide is tested for its ability to increase [Ca²⁺[ᵢ in ciliated tracheal cells and to induce ciliary damage in tracheal epithelia. Membrane preparations isolated from nonpathogenic Mhyo (strain J) are used as negative controls in these experiments. Tracheal epithelial cells in inserts are treated with one of the following seven treatments: (1) negative controls (e.g., membrane preparation from nonpathogenic Mhyo (strain J), 100 μg/mL), (2) positive controls (e.g., membrane preparation from Mhyo Strain 91-3, 100 μg/mL), (3) soluble tryptic Mhyo polypeptide fragments (10 μg/mL), (4) recombinant Mhyo polypeptide (0.1 μg/mL), (5) recombinant Mhyo polypeptide (1 μg/mL), (6) recombinant Mhyo polypeptide (10 μg/mL), and (7) recombinant Mhyo polypeptide (100 μg/mL). Each condition is performed in triplicate with the entire experiment being repeated at least three times. The [Ca²⁺]ᵢ determinations are performed as described above.

The following techniques are used to assess adherence, cilia damage, and cilia loss. Enzyme-digested epithelial cells prepared using a sterile technique are plated at a concentration of 4-5×10⁵ cells/cm² onto Millicell-PCF inserts (0.45 μm pore size, 0.6 cm² area, Millipore, Bedford, Mass.) as described elsewhere (Young et al., Vet. Microbiol., 71:269-279 (2000)). The inserts are coated with human placental collagen and placed in 24-well culture plates. The cells are grown on the air-liquid interface and nourished from underneath with serum-free DMEM/F-12 (1:1) containing 2% ultroser G serum substitute (USG medium) supplemented with penicillin and streptomycin.

Ciliated tracheal epithelial cell cultures after 18-22 days of growth are used. The culture medium is discarded and replaced with fresh DMEM/F-12 medium containing untreated Mhyo membrane protein or recombinant Mhyo polypeptide, and incubated at 37° C., 7.2% CO₂ for either 90 minutes (for the determination of adherence and cilia damage) or two days (for the determination of cilia loss). After incubation, the inserts are washed with PBS three times to remove the unattached mycoplasmas. Cells are dissociated from the insert using trypsin-EDTA and washed with PBS. These cells are fixed in situ with glutaraldehyde and paraformaldehyde and subjected to scanning electron microscopy as previously described (Young et al., Vet. Microbiol., 71:269-279 (2000)) to determine the adherence of Mhyo to ciliated cells and the extent of cilia damage and loss. Photographs are taken from five random fields (16×23 μm²) in each sample and subjected to image analysis to obtain data for the areas occupied by cilia (for the determination of cilia loss) and the attachment of mycoplasma to the cilium.

Alternatively, adherence and cilia loss is assessed using a microtiter plate adherence assay described by Zhang et al. (Infect. Immun., 62:1616-1622 (1994)) and/or a tracheal explant model described by DeBay and Ross (Infect. Immun., 62:5312-5318 (1994)).

The active site of the Mhyo polypeptide is mapped using deletion mutagenesis and/or overlapping peptide sequences. In addition, Mhyo polypeptide preparations are used to vaccinate swine to help control swine mycoplasmal pneumonia, and/or analogs of the peptide sequences corresponding to the active site are used to block the cell's receptors for the Mhyo membrane polypeptide.

### Example 5

### Producing Antibodies Against the Mhyo Polypeptide that Induces [Ca²⁺]ᵢ Increases in Porcine Ciliated Tracheal Cells

Five female BALB/c mice (8-10 weeks old) are immunized with the purified Mhyo membrane polypeptide. The purified Mhyo polypeptide having the ability to increase [Ca²⁺ᵢ in ciliated tracheal cells is obtained via HPLC, SDS-PAGE, or other purification techniques. Each mouse is given three biweekly intraperitoneal injections of 50 μg of the polypeptide in Freud's adjuvant. A final intravenous booster of 5 μg of the polypeptide in saline is given one month after the third injection and 3 days prior to fusion with the SP2/0 myeloma cells. About 500 separate clones are screened during each fusion, and all 5 mice are used to generate MAbs. Hybridoma screening is performed using an indirect ELISA by coating an ELISA plate with purified Mhyo membrane polypeptide along with control membrane polypeptide of M. flocculare and nonpathogenic Mhyo (strain J). A goat anti-mouse IgG-horseradish peroxidase conjugate is used to detect MAbs.

### Example 6

### Identifying Antibodies that Inhibit Mhyo Polypeptide-Induced [Ca²⁺]ᵢ Increases in Porcine Ciliated Tracheal Cells

Antibodies at different dilutions are added to the mycoplasma membrane preparation or to the purified Mhyo polypeptide prior to inclusion in the [Ca²⁺]ᵢ determinations. The antibodies also are added to antigen-free samples to control for nonspecific antibody effects. The methods for testing changes in [Ca²⁺]ᵢ is as described above.

The following techniques are used to assess the antibodies ability to inhibit Mhyo adherence and Mhyo-induced cilia damage and cilia loss. The polyclonal and monoclonal antibodies exhibiting the ability to block Mhyo- and recombinant Mhyo polypeptide-induced increase in [Ca²⁺]ᵢ in ciliated cells are used. Tracheal epithelial cells in the inserts are treated with one of the following treatments: (1) controls, (2) Mhyo strain 91-3 (10⁹ CCU), (3) antibody preparation (dilution A) plus Mhyo strain 91-3, (5) antibody preparation (dilution B) plus Mhyo strain 91-3, (6) antibody preparation (dilution C) plus Mhyo strain 91-3, (7) antibody preparation (dilution D) plus Mhyo strain 91-3. The antibodies are added to Mhyo-free samples to control for nonspecific antibody effects. In addition, heat-inactivate antibodies are used to confirm that heating abolishes a specific inhibition of Mhyo-induced adherence and cilia loss. Each condition is performed in triplicate with the entire experiment being repeated at least three times.

Data for the areas occupied by cilia and attachment of Mhyo to cilia are analyzed using ANOVA, and mean comparisons are performed using Tukey's test. The α level is set at P≦0.05.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A substantially pure polypeptide, wherein said polypeptide increases calcium release from porcine ciliated tracheal cells, and wherein the molecular weight of said polypeptide is between about 30 kDa and about 150 kDa.

2. The polypeptide of claim 1, wherein said polypeptide is a mycoplasma polypeptide.

3. The polypeptide of claim 1, wherein said polypeptide is obtained from pathogenic Mycoplasma hyopneumoniae.

4. The polypeptide of claim 1, wherein said polypeptide is about 80 percent pure.

5. The polypeptide of claim 1, wherein said polypeptide is about 90 percent pure.

6. The polypeptide of claim 1, wherein the molecular weight of said polypeptide is about 30 kDa.

7. The polypeptide of claim 1, wherein the molecular weight of said polypeptide is about 60 kDa.

8. The polypeptide of claim 1, wherein the molecular weight of said polypeptide is about 65 kDa.

9. The polypeptide of claim 1, wherein the molecular weight of said polypeptide is about 90 kDa.

10. The polypeptide of claim 1, wherein the molecular weight of said polypeptide is about 120 kDa.

11. The polypeptide of claim 1, wherein said polypeptide is a tryptic fragment.

12. The polypeptide of claim 1, wherein the molecular weight of said polypeptide following a tryptic digest is about 35 kDa.

13. The polypeptide of claim 1, wherein the molecular weight of said polypeptide following a tryptic digest is about 50 kDa.

14. A substantially pure antibody capable of binding a polypeptide, wherein said polypeptide increases calcium release from porcine ciliated tracheal cells, and wherein the molecular weight of said polypeptide is between about 30 kDa and about 150 kDa.

15. The antibody of claim 14, wherein said antibody is a monoclonal antibody.

16. The antibody of claim 14, wherein said antibody is a mouse antibody.

17. The antibody of claim 14, wherein said polypeptide is a tryptic fragment.

18. The antibody of claim 14, wherein said polypeptide is a mycoplasma polypeptide.

19. The antibody of claim 14, wherein said polypeptide is obtained from pathogenic Mycoplasma hyopneumoniae.

20. The antibody of claim 14, wherein said antibody is about 80 percent pure.

21. The antibody of claim 14, wherein said antibody is about 90 percent pure.

22. A method for inducing an immune response in a mammal, wherein said immune response is against a mycoplasma polypeptide, said method comprising administering a substantially pure mycoplasma polypeptide to said mammal under conditions wherein said mammal produces antibodies against said polypeptide, wherein said polypeptide increases calcium release from porcine ciliated tracheal cells, and wherein the molecular weight of said polypeptide is between about 30 kDa and about 150 kDa.

23. The method of claim 22, wherein said mammal is a mouse, rabbit, or pig.

24. A method for binding an antibody to a polypeptide, wherein said polypeptide increases calcium release from porcine ciliated tracheal cells, and wherein the molecular weight of said polypeptide is between about 30 kDa and about 150 kDa, said method comprising:
a) obtaining an antibody capable of binding said polypeptide, and b) contacting said antibody with said polypeptide under conditions wherein said antibody binds said polypeptide.

25. The method of claim 24, wherein said antibody is a monoclonal antibody.

26. The method of claim 24, wherein said antibody is a mouse antibody.

27. The method of claim 24, wherein said polypeptide is a mycoplasma polypeptide.

28. A method for identifying an inhibitor of mycoplasma induced calcium release from cells, said method comprising:
a) contacting cells with a mycoplasma polypeptide and a test compound, wherein said polypeptide increases calcium release from porcine ciliated tracheal cells, and wherein the molecular weight of said polypeptide is between about 30 kDa and about 150 kDa,
b) determining whether said test compound inhibits said cells from releasing calcium, wherein inhibition of calcium release from said cells by said test compound indicates that said test compound is said inhibitor.

29. The method of claim 28, wherein said test compound is a protease.

30. The method of claim 28, wherein said test compound is an antibody.

31. A method for identifying an inhibitor of calcium release from cells induced by a mycoplasma polypeptide, wherein said polypeptide increases calcium release from porcine ciliated tracheal cells, and wherein the molecular weight of said polypeptide is between about 30 kDa and about 150 kDa, said method comprising:
a) contacting cells with a mycoplasma polypeptide pretreated with a test compound, and
b) determining whether said test compound inhibits said cells from releasing calcium, wherein inhibition of calcium release from said cells by said test compound indicates that said test compound is said inhibitor.

32. The method of claim 31, wherein said test compound is a protease.

33. The method of claim 31, wherein said test compound is an antibody.
